(19) United States
(12) Patent Application Publication
(10) Pub. No.: US 2014/0166036 A1
(43) Pub. Date: Jun. 19, 2014

(54) TRANSFORMATION MEDIUM FOR MODELS CONTAINING KERATIN FIBERS

(71) Applicant: Henkel AG & Co. KGaA, Duesseldorf (DE)

(72) Inventors: Pamela Kaftan, Hamburg (DE); Uwe Bergemann, Hamburg (DE); Burkhard Mueller, Duesseldorf (DE)

(73) Assignee: Henkel AG & Co. KGaA, Duesseldorf (DE)

(21) Appl. No.: 14/132,052

(22) Filed: Dec. 18, 2013

(30) Foreign Application Priority Data
Dec. 19, 2012 (DE) 10 2012 223 850.6

(51) Int. Cl. A61K 8/88; A61Q 5/06

(52) U.S. Cl. CPC A61K 8/88; A61Q 5/06
USPC 132/210; 424/70.16; 424/47

(57) ABSTRACT

Cosmetic agents, apparatuses and methods for treating keratin-containing fibers, in particular human hair, are provided. In an embodiment, a cosmetic agent for treating keratin-containing fibers includes
(a) at least one setting polymer, and
(b) at least one starch compound dispersed as solid particles, and
(c) at least one solvent, different from water, that is liquid at 25° C. and 1013 mbar, and
(d) based on the total weight of the agent, about 0 to about 20 wt % water,
and is suitable for increasing the volume of and for remodeling keratin-containing fibers, in particular human hair.

### CROSS-REFERENCE TO RELATED APPLICATIONS

This application claims priority to German Application No. 10 2012 223 850.6, filed Dec. 19, 2012, which is hereby incorporated in its entirety by reference.

### TECHNICAL FIELD

The invention relates to the technical field of temporary reshaping of keratin-containing fibers, in particular human hair. It relates in particular to agents for hair treatment containing at least one setting polymer in combination with further special ingredients, to the use of said agents for the temporary deformation and remodeling of keratin-containing fibers, and furthermore to sprays resp. foams based on said agents.

### BACKGROUND

The initially most important property of an agent for temporary reshaping of keratinic fibers, hereinafter also called a “styling agent,” is to give the strongest possible hold to the treated fibers in the shape that has been generated. In addition to a high degree of hold, styling agents must meet a large number of further requirements. These can be subdivided roughly into: properties on the hair; properties of the particular formulation, e.g. properties of the foam, gel, or sprayed aerosol; and properties that relate to the handling of the styling agent, the properties on the hair being of particular importance. Moisture resistance, low tack, and a balanced conditioning effect may be mentioned in particular. In addition, a styling agent should be universally usable for, if possible, all types of hair. Very recently there have been increased efforts to develop agents for temporary deformation that, in addition to the aforementioned requirements, are additionally intended to allow a remodelable shape.

A plurality of synthetic polymers that are utilized in styling agents have already been developed in order to meet the various requirements. The polymers can be subdivided into cationic, anionic; nonionic, and amphoteric setting polymers. Ideally, upon application to the hair in even a small quantity, the polymers yield a polymer film that on the one hand imparts a strong hold to the hairstyle but on the other hand is sufficiently flexible not to break under stress. If the polymer is too brittle, this results in the formation of so-called “film plaques,” i.e. residues that detach as the hair moves and give the impression that the user of the corresponding styling agent has dandruff. In addition, hair coated with a setting polymer often feels rough.

Conventional temporary polymer-based hair setting agents for strong hold are usually not suitable for producing remodelable hairstyles. The reason for this is the high strength of the film that is formed. When hair whose shape has already been retained using the polymer is reshaped again, the film that is present usually breaks. The hairstyle that has just been created consequently cannot readily be retained using the styling agent already present on the hair. In order to avoid film breakage, the hairstyle is often remodeled under the action of heat, and the polymer film is kept flexible by the heat. This helps avoid film breakage. Polymers having a high degree of hold often have a high melting temperature, however. This in turn requires the use of high temperatures during remodeling, and the high temperatures result in additional hair damage. The latter is unconditionally to be avoided.

The object of the present invention was therefore to make available agents for the temporary deformation of keratin-containing fibers, in particular human hair, that are notable for a high degree of hold and that permit, to the greatest extent possible without the use of heat, good remodelability of the shape (e.g. hairstyle) established using said agent. It was also an object of the invention to generate a correspondingly sustainable volume for the fiber collective without having the hair feel dull. The styling agents were furthermore intended not exhibit the problems recited above.

### BRIEF SUMMARY

Cosmetic agents, apparatuses and methods for treating or reshaping keratin fibers are provided. In one embodiment, a cosmetic agent is provided for treating keratin-containing fibers. The cosmetic agent contains
(a) at least one setting polymer,
(b) at least one starch compound dispersed as solid particles,
(c) at least one solvent, different from water, that is liquid at about 25° C. and about 1013 mbar, and
(d) based on the total weight of the agent, about 0 to about 20.0 wt % water.

In another embodiment, an apparatus for reshaping keratinic fibers is provided. The apparatus includes an aerosol delivery apparatus and a composition contained therein. The composition includes, based on its weight, about 1 to about 89 wt % of a cosmetic agent containing at least one setting polymer, at least one starch compound dispersed as solid particles, at least one solvent, different from water, that is liquid at about 25° C. and about 1013 mbar, and about 0 to about 20 wt % water based on the total weight of the agent, and about 20 to about 99 wt % of at least one propellant.

Another embodiment provides a method for remodeling an imposed shape of a fiber collective of keratin-containing fibers. In the method, (i) the fibers are dry before remodeling begins, and already have a shape retained by utilization of a cosmetic agent containing at least one setting polymer, at least one starch compound dispersed as solid particles, at least one solvent, different from water, that is liquid at about 25° C. and about 1013 mbar, and about 0 to about 20 wt % water based on the total weight of the agent. Further, in the method, (ii) the fibers are brought into a new shape and (iii) the new shape is retained by means of the cosmetic agent already applied before step (i) onto the fibers in order to retain the shape. The method includes the provision that no application of a further cosmetic agent occurs in order to retain the new shape for purposes of (iii).

### DETAILED DESCRIPTION

One skilled in the art understands “remodelability” to mean the reshaping of keratin-containing fibers, in particular hair, wherein
(i) the fibers/hair are dry before reshaping begins, and already have a shape retained by the use of a cosmetic agent containing at least one setting polymer,
(ii) said fibers/hair are brought into a new shape, and
(iii) said new shape is again retained
(iv) without application, for retention according to step (iii), of a further cosmetic agent containing at least one setting polymer.

A dry shape/hairstyle, retained by means of polymers, that can be reshaped and retained without the assistance of a further cosmetic agent containing at least one setting polymer is accordingly a “remodelable” shape/hairstyle.

“Dry” is understood to mean in no way dripping wet, but instead the state in which the liquid residues (e.g. water, organic solvents) adhering to the fibers have evaporated to the extent that the moisture content of the fibers is substantially in equilibrium with the moisture in the air, or the fiber absorbs moisture from the ambient air.

“Setting polymers” contribute to the hold, and/or to buildup of the hair volume and hair fullness, of the overall hairstyle. These polymers are at the same time also film-forming polymers and are therefore generally typical substances for shape-imparting hair-treatment agents such as hair setting agents, hair foams, hair waxes, hair sprays. It is certainly possible for film formation to be localized, and for only a few fibers to be connected to one another. “Film-forming polymers” are furthermore understood according to the present invention as those polymers which, when utilized in a 0.01 to 20 wt % aqueous, alcoholic, or aqueous alcoholic solution, are capable of depositing a transparent polymer film onto the hair. The so-called “curl retention” test is often used as a test method for the setting effect of a polymer.

All indications as to quantity ranges are always understood for purposes of the invention to include the respectively recited upper and lower limit.

According to all formulas, a chemical bond labeled with the symbol * denotes a free valence of a structural fragment. In a corresponding polymer, further structural fragments bond to this free valence.

It has been found that by using the agent of the first subject of the invention, keratin-containing fibers, in particular human hair, can be brought into a remodelable shape, in particular a hairstyle. The shape has a strong hold and can be remodeled without the use of heat or moistening, for example using the hands or a comb. Although the use of heat and/or moisture is disadvantageous because of the possible fiber damage due to heat and because of the additional method steps to be carried out, the agent according to the present invention nevertheless permits the utilization of heat and/or, in moderation, moisture when reshaping a shape imposed using said agent. The “remodeling” utilization form is suitable in particular for increasing the volume of a fiber collective (for example, volume styling for head hair).

The agent according to the present invention contains as an obligatory component (a) at least one setting polymer. It is preferred in turn if said setting polymer is selected from one or more representatives of the group that is constituted from anionic setting polymers, amphoteric setting polymers, temporarily cationic setting polymers, and nonionic setting polymers.

“Temporarily cationic” polymers usually contain, as a temporarily cationic group, at least one amino substituent that, in cosmetic utilization conditions in water, is present as a protonated ammonium group and thus cationically. The degree of cationization depends on pH. Said polymers are free of permanently cationic groups such as, for example, ammonium substituents, and are free of structural units having anionic groups that must be compensated for with counter ions while maintaining electroneutrality.

The setting polymers are contained in the agents according to the present invention preferably in a quantity from 0.1 wt % to 20.0 wt %, particularly preferably from about 0.2 wt % to about 15.0 wt %, very particularly preferably from about 0.5 wt % to about 10.0 wt %, based in each case on the weight of the agent according to the present invention.

In the context of a preferred embodiment, the agent according to the present invention contains as said setting polymer at least one anionic setting polymer comprising at least one structural unit of formula (I) and at least one structural unit of formula (II)

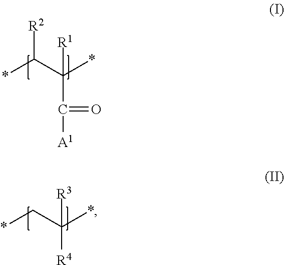

in which
R¹ and R² mutually independently denote a hydrogen atom or a methyl group, with the provision that R¹ and R² do not simultaneously denote a methyl group,
R³ denotes a hydrogen atom or a methyl group,
R⁴ denotes a carbamoyl group, a linear or branched (C₄ to C₁₂) alkylaminocarbonyl group, a linear or branched (C₄ to C₁₂) alkoxylcarbonyl group, a linear or branched (C₂ to C₁₂) acyloxy group, a (C₂ to C₄) hydroxyalkylcarbonyl group, or a phenyl group,
A¹ denotes a hydroxy group or an organic residue having at least one sulfonic acid group, which binds to the structural unit via an oxygen atom or an NH group.

It is particularly preferred according to the present invention if the anionic setting polymer contains at least one structural unit of formula (I) that is selected from at least one structural unit of formulas (I-1) to (I-5)

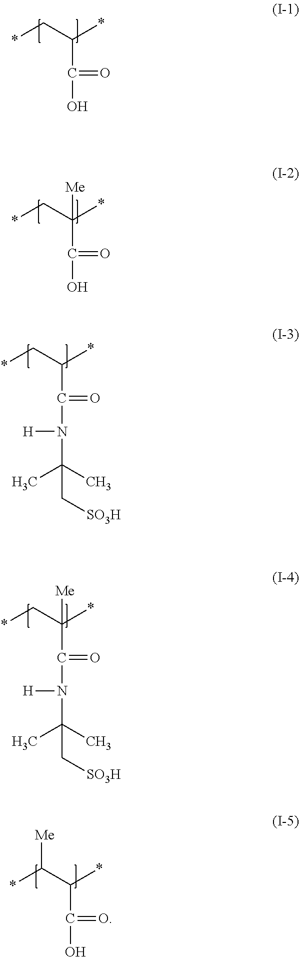

It is particularly preferred according to the present invention if the anionic setting polymer contains at least one structural unit of formula (II) that is selected from at least one structural unit of formulas (II-1) to (II-7)

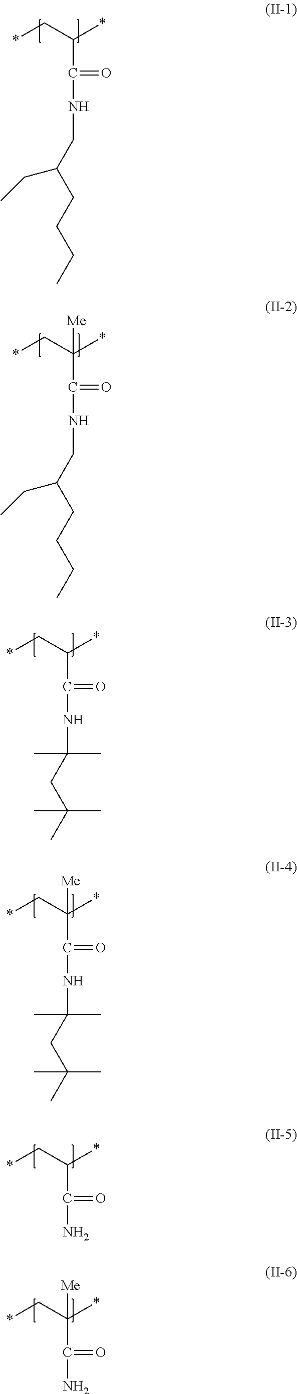

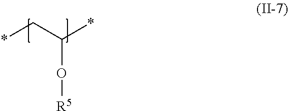

in which
R⁵ denotes a (C₂ to C₁₂) acyl group (in particular acetyl or neodecanoyl).

It is furthermore preferred if the setting polymer additionally contains, besides the above structural units of formulas (I) and (II), at least one structural unit of formula (III)

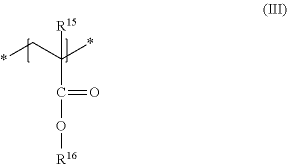

in which
R¹⁵ denotes a hydrogen atom or a methyl group,
R¹⁶ denotes a (C₁ to C₃) alkyl group (in particular a methyl group or an ethyl group).

In general, it is particularly preferred if, for all the preceding and also subsequent embodiments, the group A¹ of the structural unit of formula (I) is present in entirely or partly neutralized fashion. Preferred agents according to the present invention therefore additionally contain at least one alkanolamine. The alkanolamines usable as alkalizing agents according to the present invention are preferably selected from primary amines having a C₂ to C₆ alkyl basic structure that carries at least one hydroxyl group. Particularly preferred alkanolamines are selected from the group that is constituted from 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Alkanolamines very particularly preferred according to the present invention are selected from the group of: 2-aminoethan-1-ol, 2-amino-2-methylpropan-1-ol, and 2-amino-2-methylpropane-1,3-diol.

In the context of a first preferred embodiment, the agent according to the present invention contains at least one anionic setting polymer that comprises at least one structural unit of formula (I-2), at least one structural unit of formula (II), and at least one structural unit of formula (III)

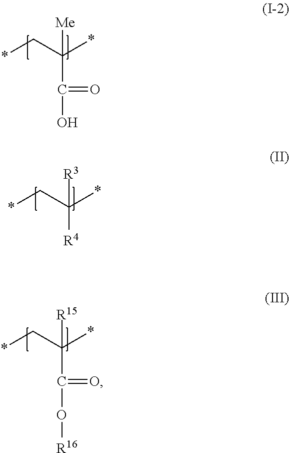

in which
R³ and R¹⁵ mutually independently denote a hydrogen atom or a methyl group,
R⁴ denotes a branched (C₄ to C₈) alkoxycarbonyl group, a linear (C₄ to C₈) alkoxycarbonyl group, a branched (C₄ to C₈) alkylaminocarbonyl group, a linear (C₄ to C₈) alkylaminocarbonyl group, or a phenyl group, and
R¹⁶ denotes a (C₁ to C₃) alkyl group (in particular a methyl group or an ethyl group).

Copolymers of methacrylic acid and ethyl acrylate and tert-butyl acrylate are suitable as particularly preferred. Such copolymers are obtainable, for example, from the BASF SE company under the commercial names Luvimer 100 P, Luvimer 30 E, or Luvimer 36 D.

It is particularly preferred in this context if, for all embodiments of this first preferred embodiment, the structural unit of formula (I-2) is present in entirely or partly neutralized fashion. Preferred agents according to the present invention therefore additionally contain at least one alkanolamine. The statements made above (vide supra) apply with regard to suitable alkalizing agents according to the present invention.

In the context of a second preferred embodiment, the agent according to the present invention contains at least one anionic setting polymer that comprises at least one structural unit of formula (I-1), at least one structural unit of formula (II), and at least one structural unit of formula (III)

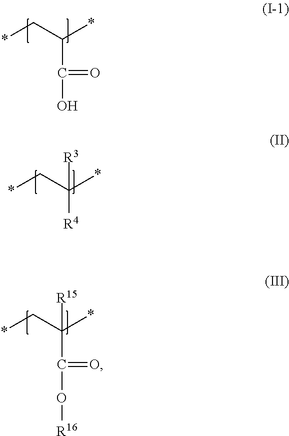

in which
R³ and R¹⁵ mutually independently denote a hydrogen atom or a methyl group, R⁴ denotes a branched (C₄ to C₈) alkylaminocarbonyl group, a linear (C₄ to C₈) alkylaminocarbonyl group, or a phenyl group, and
R¹⁶ denotes a (C₁ to C₃) alkyl group (in particular a methyl group or an ethyl group).

It is particularly preferred in this context if, for all embodiments of this second preferred embodiment, the structural unit of formula (I-1) is present in entirely or partly neutralized fashion. Preferred agents according to the present invention therefore additionally contain at least one alkanolamine. The statements made above apply (vide supra) with regard to suitable alkalizing agents according to the present invention.

Copolymers of acrylic acid and ethyl acrylate and tert-butylacrylamide are suitable as particularly preferred. Such copolymers are obtainable, for example, from the BASF SE company under the commercial names Ultrahold Strong or Ultrahold 8.

In the context of a third embodiment, those agents which contain as an anionic setting polymer at least one polymer that contains at least one structural unit of formula (I-3) and at least one structural unit of formula (II-5)

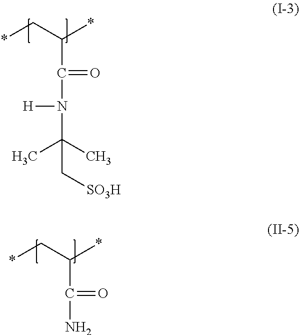

are considered preferred according to the present invention.

Preferred anionic setting polymers of this kind are selected from at least one polymer of the group that is constituted from
copolymers of 2-acrylamido-2-methylpropanesulfonic acid and acrylamide,
copolymers of 2-acrylamido-2-methylpropanesulfonic acid, acrylamide, and acrylic acid.

An agent of this embodiment which is particularly preferred according to the present invention is characterized in that it contains as an anionic setting polymer a copolymer (a1).

These copolymers (a1) can be described by the general formula

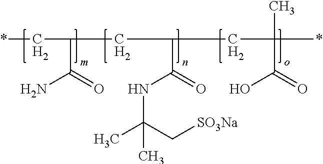

where the indices m, n and o each vary depending on the molar mass of the polymer and are not intended to signify that these are block copolymers. Structural units can instead be present in statistically distributed fashion in the molecule.

Particularly preferred agents according to the present invention are characterized in that the copolymer (b1) has a molar mass from about 50 to about 500 kDa, preferably from about 100 to about 450 kDa, more preferably from about 150 to about 400 kDa, and in particular from about 200 to about 300 kDa.

It is particularly preferred in this context if, for all embodiments of this third preferred embodiment, the structural unit of formula (I-3) resp. (I-2) is present in entirely or partly neutralized fashion. Preferred agents according to the present invention therefore additionally contain at least one alkanolamine. The alkalizing agents recited above (vide supra) are considered preferably suitable according to the present invention.

Copolymers of acrylamide with methacrylic acid and acryloyldimethyl taurate are obtainable, for example, under the commercial name Acudyne® SCP (Rohm & Haas).

In the context of a fourth embodiment, those agents which contain as an anionic setting polymer at least one polymer that contains at least one structural unit of formula (I-5) and at least one structural unit of formula (II-7)

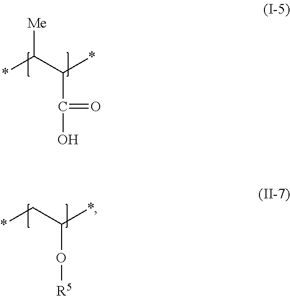

in which R⁵ denotes a (C₂ to C₁₂) acyl group (in particular acetyl or neodecanoyl), are considered preferred according to the present invention.

Particularly preferred polymers (b) of this kind are selected from at least one polymer of the group that is constituted from

copolymers of vinyl acetate and crotonic acid,

copolymers of vinyl propionate and crotonic acid,

copolymers of vinyl neodecanoate, vinyl acetate, and crotonic acid.

Such copolymers are made available, for example, by the Clariant company under the commercial name Aristoflex A 60 (INCI name: VA/Crotonates Copolymer) in an isopropanol/water mixture (60 wt % active substance), by the BASF company under the commercial name Luviset CA 66 (vinyl acetate/crotonic acid copolymer 90:10, INCI name: VA/Crotonates Copolymer), and by the National Starch company under the commercial name Resyn 28-2942 resp. Resyn 28-2930 (INCI name: VA/Crotonates/Vinyl Neodecanoate Copolymer).

It is particularly preferred in this context if, for all embodiments of this fourth embodiment, the structural unit of formula (I-5) is present in entirely or partly neutralized fashion. Preferred agents according to the present invention therefore additionally contain at least one alkanolamine. The alkalizing agents recited above (vide supra) are considered preferably suitable according to the present invention.

In the context of a preferred embodiment, the agent according to the present invention contains as said setting polymer at least one amphoteric setting polymer comprising at least one structural unit of formula (I) and at least one structural unit of formula (II)

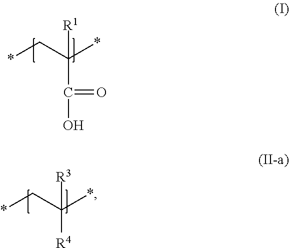

in which
R¹ and R³ mutually independently denote a hydrogen atom or a methyl group,
R⁴ denotes a linear or branched (C₄ to C₁₂) alkylaminoethylaminocarbonyl group, a linear or branched (C₄ to C₁₂) alkylaminopropylaminocarbonyl group, a linear or branched (C₄ to C₁₂) alkylaminoethyloxycarbonyl group, a linear or branched (C₄ to C₁₂) alkylaminopropyloxycarbonyl group.

It is particularly preferred in this context if, for all embodiments of this embodiment and subsequent preferred forms thereof, the structural unit of formula (I) is present in entirely or partly neutralized fashion. Preferred agents according to the present invention therefore additionally contain at least one alkanolamine. The alkalizing agents recited above (vide supra) are considered preferably suitable according to the present invention.

At least one amphoteric setting polymer that additionally contains, besides respectively at least one of the above structural units of formulas (I) and (II-a), at least one structural unit of formula (III)

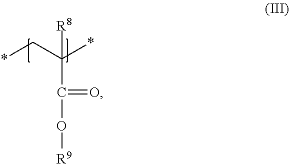

in which
R⁸ denotes a hydrogen atom or a methyl group,
R⁹ denotes a (C₁ to C₄) alkyl group (in particular a methyl group or an ethyl group), is particularly suitable.

A particularly preferred amphoteric setting polymer contains, as a preferred structural unit of formula (II-a), at least one structural unit selected from at least one structural unit from the group that is constituted from structural units of formulas (II-8) to (II-15)

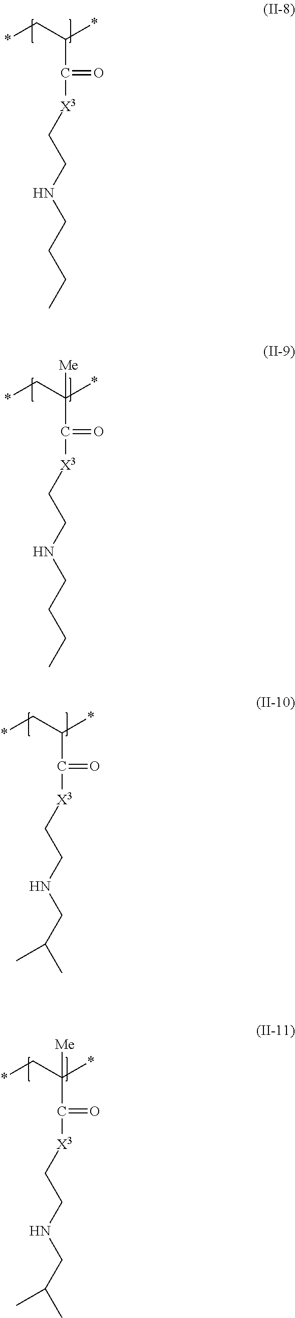

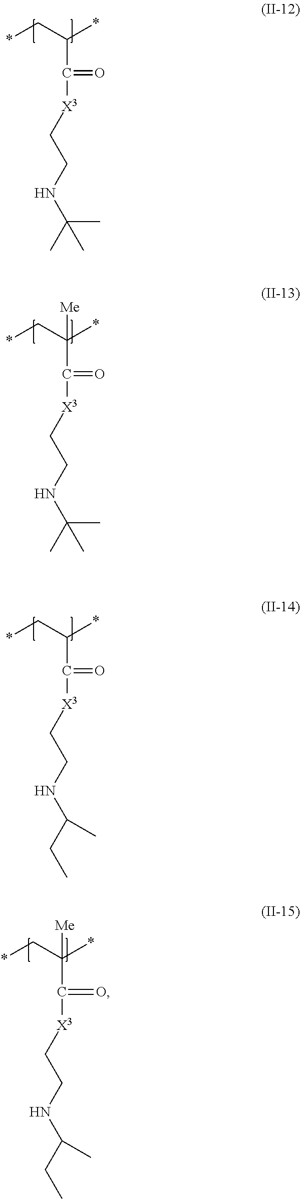

in which
X³ denotes an oxygen atom or an NH group. It is preferred according to the present invention if X³ according to formulas (II-8) to (II-15) denotes an oxygen atom.

In the context of a preferred embodiment of the invention, the agent according to the present invention contains at least one amphoteric setting polymer that comprises at least one structural unit of formula (I-1), at least one structural unit of formula (II-3), and at least one structural unit of formula (II-16) (selected in particular from the group that is constituted from the above formulas (II-8) to (II-15) with the provision that X³ denotes an oxygen atom)

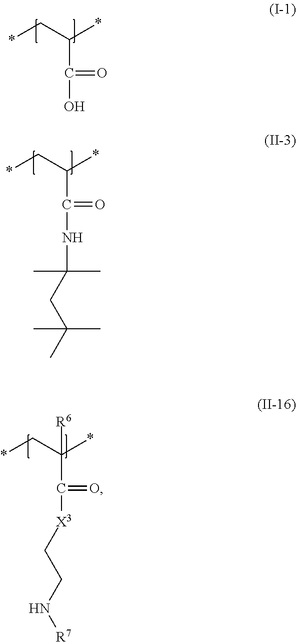

in which X³ denotes an oxygen atom or an NH group,
R⁶ denotes a hydrogen atom or a methyl group, and
R⁷ denotes an alkyl group having 4 carbon atoms (in particular n-butyl, sec-butyl, isobutyl, or tert-butyl).

It is in turn particularly preferred if the amphoteric setting polymer additionally contains, besides the above structural units of formulas (I-1), (II-3), and (II-16), at least one structural unit of formula (III)

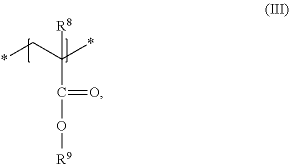

in which
R⁸ denotes a hydrogen atom or a methyl group, and
R⁹ denotes a (C₁ to C₄) alkyl group (in particular a methyl group or an ethyl group).

Preferred amphoteric setting polymers of this kind are selected from the group that is constituted from:
copolymers of acrylic acid, at least one (C₁ to C₄) alkyl acrylate, at least one C₄ alkylaminoethyl methacrylate, and at least one C₈ alkylacrylamide.

An example of an amphoteric setting polymer particularly preferably usable in the context of this embodiment is the polymer, obtainable from the National Starch company under the commercial name Amphomer® 028-4910, having the INCI name Octylacrylamide/Acrylates/Butylaminoethyl Methacrylate Copolymer.

Homo- or copolymers that are constructed from at least one of the following monomers are suitable as preferred nonionic setting polymers: N-vinylpyrrolidone, N-vinylcaprolactam, vinyl esters (such as e.g. vinyl acetate, vinyl alcohol), acrylamide, methacrylamide, alkyl- and dialkylacrylamide (in particular N-methyl- and N,N-dimethylacrylamide), alkyl- and dialkylmethacrylamide (in particular N-methyl- and N,N-dimethylmethacrylamide), alkyl acrylate, alkyl methacrylate, wherein the alkyl groups of these monomers are selected in each case from (C₁ to C₃) alkyl groups.

Nonionic setting polymers that are particularly suitable for the agent according to the present invention contain at least one of the following structural units

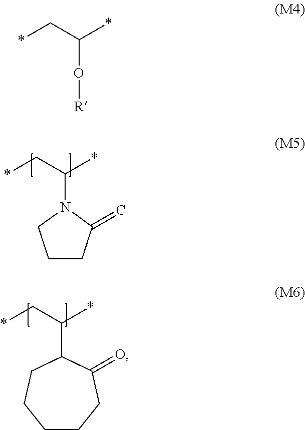

in which R′ denotes a hydrogen atom or a (C₁ to C₃₀) acyl group, in particular a hydrogen atom or an acetyl group.

Homopolymers of vinylcaprolactam or of vinylpyrrolidone (for example, Luviskol® K 90 or Luviskol® K 85 of the BASF SE company), copolymerizates of vinylpyrrolidone and vinyl acetate (in which context it is in turn preferred if the molar ratio of the structural units from the N-vinylpyrrolidone monomer to the structural units from the vinyl acetate monomer that are contained in the polymer is in the range from 20 to 80 to 80 to 20, in particular from 30 to 70 to 60 to 40; marketed, for example, under the trademark Luviskol® VA 37, Luviskol® VA 55, Luviskol® VA 64, and Luviskol® VA 73 from the BASF SE company), terpolymers of vinylpyrrolidone, vinyl acetate, and vinyl propionate, polyacrylamides (for example Akypomine® P 191 of the CHEM-Y company), polyvinyl alcohols (marketed, for example, under the commercial names Elvanol® of Du Pont or Vinol® 523/540 of the Air Products company) are particularly suitable.

Agents that contain as a nonionic setting polymer at least one polymer selected from the group that is constituted from
polyvinylpyrrolidone,
copolymers of N-vinylpyrrolidone and vinyl esters of carboxylic acids having 2 to 18 carbon atoms, in particular of N-vinylpyrrolidone and vinyl acetate,
   are preferred according to the present invention.

Particularly preferred agents are characterized in that the temporarily cationic setting polymer comprises at least one structural unit of formula (M1) and at least one structural unit of formula (M2) and at least one structural unit of formula (M3)

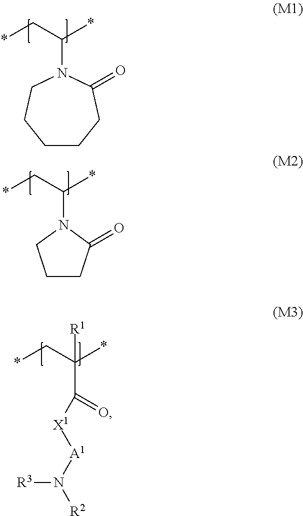

R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R⁴ mutually independently denote a (C₁ to C₄) alkyl group.

R¹ in formula (M3) preferably denotes a methyl group.

Examples of (C₁ to C₄) alkyl groups according to formula (M3) are methyl, ethyl, isopropyl, n-propyl, n-butyl, sec-butyl, isobutyl, and tert-butyl. R² and R³ in formula (M3) preferably denote a methyl group.

Very particularly suitable polymers of this embodiment have at least one structural unit of formula (M3) in which A¹ denotes an ethane-1,2-diyl group and X¹ denotes an oxygen atom. Preferably in turn, a polymer of this kind preferably comprises at least one structural unit of formula (M1) and at least one structural unit of formula (M2) and at least one structural unit of formula (M3-1)

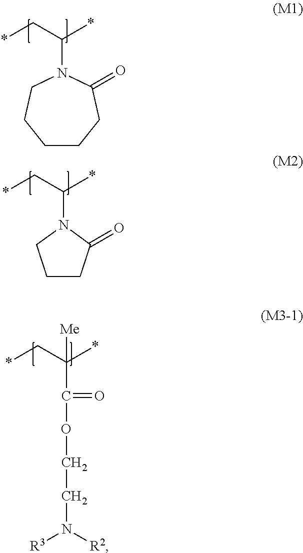

in which R² and R³ mutually independently denote a (C₁ to C₄) alkyl group, in particular methyl. Examples of (C₁ to C₄) alkyl groups according to formula (M3-1) are methyl, ethyl, isopropyl, n-propyl, n-butyl, sec-butyl, isobutyl, and tert-butyl.

Very particularly preferably, the agent according to the present invention contains as component (a) of the agent according to the present invention a terpolymer of N-vinylcaprolactam, N-vinylpyrrolidone, and N,N-dimethylaminoethyl methacrylate. Such copolymers can be obtained, for example, from the ISP company under the trade name Advantage LC-E (INCI name: Vinylcaprolactam/VP/Dimethylaminoethyl Methacrylate Copolymer, Lauryl Pyrrolidone; 37 wt % active substance in ethanol with added N-laurylpyrrolidone), Advantage S (INCI name: Vinylcaprolactam/VP/Dimethylaminoethyl Methacrylate Copolymer, 100 wt % active substance, powder) or Advantage LC-A (INCI name: Vinylcaprolactam/VP/Dimethylaminoethyl Methacrylate Copolymer; 37 wt % active substance in ethanol).

It is particularly preferred if the cosmetic agent according to the present invention contains as a setting polymer at least a combination of
(i) at least one temporarily cationic polymer comprising at least one structural unit of formula (M1) and at least one structural unit of formula (M2) and at least one structural unit of formula (M3)

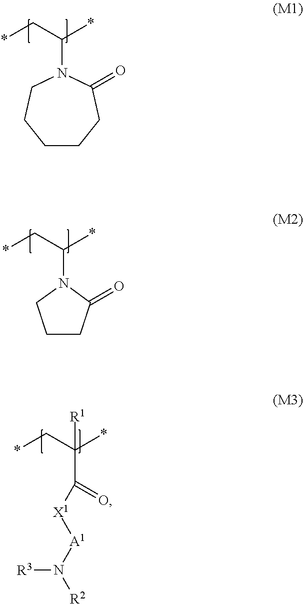

R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R³ mutually independently denote a (C₁ to C₄) alkyl group,
and
(ii) at least one amphoteric setting polymer comprising at least one structural unit of formula (I) and at least one structural unit of formula (II)

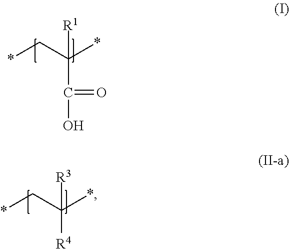

in which
R¹ and R³ mutually independently denote a hydrogen atom or a methyl group,
R⁴ denotes a linear or branched (C₄ to C₁₂) alkylaminoethylaminocarbonyl group, a linear or branched (C₄ to C₁₂) alkylaminopropylaminocarbonyl group, a linear or branched (C₄ to C₁₂) alkylaminoethyloxycarbonyl group, a linear or branched (C₄ to C₁₂) alkylaminopropyloxycarbonyl group.
   A combination of the respective preferred embodiments of polymers (i) and (ii) of the aforementioned combination is in turn preferred (vide supra).

The agent according to the present invention obligatorily contains as component (b) at least one starch compound dispersed as solid particles. This starch compound is different from the setting polymers of component (a) of the agent according to the present invention.

“Solid particles” are understood as solids that are present in particulate form at about 25° C. and about 1013 mbar.

Starch is a reserve carbohydrate that is stored by many plants in the form of starch grains (granules), usually about 1 to about 200 μm in size, in various parts of the plant, for example in tubers or roots, cereal seeds, fruits and in the pith. Starch belongs to the homoglycan family and is a polycondensation product of D-glucose. Starch is made up of three structurally different polymers of d-glucopyranose, namely amylose, amylopectin, and a so-called intermediate fraction. Higher plants contain 0 to 45 wt % amylose, based on dry substance. The intermediate fraction, which is also referred to as “anomalous amylopectin,” is structurally intermediate between amylose and amylopectin. The quantitative indications defined in the context of this Application for amylopectin encompass the intermediate fraction. Amylose is made up of predominantly linear α-1,4-glycosidically linked d-glucose, Mᵣ 50,000 to 150,000. The resulting chains form double helices in the starch. Amylopectin also contains, besides the α-1,4 links described for amylose, α-1,6 bonds (in a quantity from 4 to 6%) as branching points. The average spacing between the branching points is approximately 12 to 17 glucose units. The molar mass of 10⁷ to 7*10⁸ corresponds to approx. 10⁵ glucose units, making amylopectin one of the largest biopolymers. The aforesaid branching points are distributed over the molecule in such a way that a bundle structure, with relatively short side chains, develops. Each double helix is formed by two of these side chains. As a result of the many branching points, amylopectin is relatively easily soluble in water.

A starch compound according to the present invention is defined as starch, as well as a polycondensation product of D-glucose obtained from starch, or derivative thereof, which is modified with further chemical groups at the OH groups of the polycondensation product of D-glucose. Chemical compounds such as propylene oxide, ethylene oxide, 2-octylsuccinic acid (CAS no.: 2530-32-7) are preferably suitable for modifying said OH groups.

An example of a preferred modified starch compound is aluminum starch octenylsuccinate, an aluminum salt of a corn starch modified with 2-octylsuccinic acid, obtainable for example from the National Starch company under the commercial name DryFlo Plus resp. DryFlo Pure.

A starch modified with propylene oxide which has an average molecular weight (weight average) from 50 to 2500 kDa is likewise an example of a modified starch compound. The corresponding propylene oxide-modified starches are made available, for example, by reacting a natural starch with propylene oxide.

A starch compound preferably usable according to the present invention is selected from at least one (optionally modified) polycondensation product of D-glucose obtained from the starch of potatoes, corn, rice, peas, acorns, chestnuts, barley, wheat, bananas, sago, millet, sorghum, oats, rye, beans, yams, arrowroot or cassava. Particularly preferably, the agent according to the present invention contains at least one starch compound that is or derives from tapioca starch, potato starch, corn starch, or rice starch. Mixtures of the aforementioned starch compounds are likewise comprised under the present invention. Very particularly preferably, the starch compound is rice starch.

It is preferred according to the present invention if the solid particles of the starch compound have an average particle diameter from about 5 to about 100 μm, in particular from about 10 to about 75 μm.

The starch compound dispersed as solid particles is contained in the agents according to the present invention preferably in a quantity from about 1 to about 50 wt %, in particular from about 1.5 to about 35 wt %, based in each case on the total weight of the agent.

The agents according to the present invention contain as an obligatory component (c) at least one solvent, different from water, that is liquid at about 25° C. and about 1013 mbar.

It is preferred according to the present invention to use as a solvent of component (c) of the agent according to the present invention at least one (C₂ to C₆) alkyl alcohol having at least one hydroxy group. In the context of a particularly preferred embodiment of the agent according to the present invention, the agent contains as a solvent of component (c) at least one alcohol that contains 2 to 6 carbon atoms and 1 to 3 hydroxyl groups.

The aforesaid solvent of component (c) is preferably selected from at least one compound of the group that is constituted from ethanol, ethylene glycol, isopropanol, 1,2-propylene glycol, 1,3-propylene glycol, glycerol, n-butanol, 1,3-butylene glycol. A very particularly preferred solvent is ethanol.

Further particularly preferred solvents are polyethylene glycol and/or polypropylene glycol.

The addition in particular of polyethylene glycol and/or polypropylene glycol increases the flexibility of the polymer film formed when the agent according to the present invention is utilized. If a flexible hold is desired, the agents according to the present invention therefore preferably contain about 0.01 to about 30 wt % polyethylene glycol and/or polypropylene glycol, based on the total agent.

Very particularly preferred agents according to the present invention are characterized in that they contain said solvents (in particular the solvents recited previously as preferred), based in each case on the total weight of the agent according to the present invention, in a quantity from about 49.9 to about 98.9 wt %, preferably about 60 to about 95 wt %.

It is furthermore preferred according to the present invention if the cosmetic agent of the present invention contains, based in each case on its total weight, about 0 to about 15.0 wt %, in particular about 0 to about 10.0 wt %, most preferably about 0 to about 5.0 wt % water.

If the agent according to the present invention contains, based in each case on its total weight, water in a quantity between about 1.0 and about 20.0 wt %, preferably between about 1.0 and about 10.0 wt % water, particularly preferably between about 1.0 and about 5.0 wt % water, it is preferred according to the present invention to add to the agent according to the present invention at least one (C₈ to C₂₀) monocarboxylic acid compound of formula (MC-1)

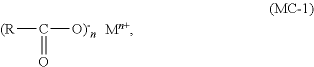

in which R denotes a (C₇ to C₁₉) alkyl group, Mⁿ⁺ denotes an n-valent cation, and
n denotes the number 1, 2, or 3 (in particular 2).
   It is preferred in turn according to the present invention to use as a setting polymer at least one amphoteric setting polymer, in particular at least one according to claim 4 or 5.

Those compounds in which, according to formula (MC-1), n signifies the number 2 have proven to be preferably suitable according to the present invention. The effect according to the present invention is particularly pronounced when, according to formula (MC-1), n denotes 2 and additionally a liquid, alcoholic carrier is present.

The group Mⁿ⁺ of formula (MC-1) denotes in general an n-valent cation. The latter serves, for reasons of electroneutrality, to compensate for the single negative charge of the carboxyl group according to formula (MC-1). Appropriate n-valent cations Mⁿ⁺ are, in principle, all mono-, di-, or trivalent cations (n=1, 2, or 3). In particular, Mⁿ⁺ according to formula (MC-1) is preferably selected from among metal cations of the physiologically acceptable metals of groups Ia, Ib, IIa, IIb, Mb, VIa, or VIII of the periodic system of the elements, ammonium ions, as well as cationic organic compounds having a quaternized nitrogen atom. The latter are obtained, for example, by protonating primary, secondary, or tertiary organic amines with an acid, for example with compounds of formula (MC-1) in their acid form (Mⁿ⁺═H⁺), or by permanently quaternizing said organic amines Examples of these cationic organic ammonium compounds are 2-ammonioethanol and 2-trimethylammonioethanol. Mⁿ⁺ according to formula (MC-1) preferably denotes an ammonium ion (n=1), an alkali metal ion (n=1) (in particular K⁺), an alkaline earth metal ion (n=2) (in particular Ca²⁺, Mg²⁺), or a zinc ion (n=2, i.e. Zn²⁺), particularly preferably a magnesium ion (Mg²⁺) or a zinc ion (Zn²⁺).

R according to formula (MC-1) preferably denotes a linear or branched (C₇ to C₂₀) alkyl group, in particular n-heptyl, n-nonyl, n-undecyl, n-tridecyl, n-pentadecyl, or n-heptadecyl.

Very particularly preferably, at least one (C₈ to C₂₀) monocarboxylic acid compound of formula (MC-1) selected from among ammonium stearate, calcium distearate, magnesium distearate, zinc distearate, ammonium isostearate, calcium diisostearate, magnesium diisostearate, zinc diisostearate, ammonium palmitate, calcium dipalmitate, magnesium dipalmitate, zinc dipalmitate, or mixtures thereof, magnesium distearate, zinc distearate, or mixtures thereof being very particularly preferably suitable.

The (C₈ to C₂₀) monocarboxylic acid compounds of formula (MC-1) according to the present invention are contained in the agent according to the present invention preferably in a quantity from 0.01 to 5.0 wt %, particularly preferably from 0.05 to 1.0 wt %, based in each case on the weight of the agent according to the present invention.

The following embodiments (A) to (R) represent particularly preferred embodiments of the invention:

(A): An agent for treating keratinic fibers, in particular human hair, containing
(a) at least one anionic setting polymer comprising at least one structural unit of formula (I) and at least one structural unit of formula (II)

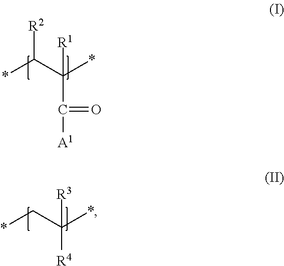

in which
R¹ and R² mutually independently denote a hydrogen atom or a methyl group, with the provision that R¹ and R² do not simultaneously denote a methyl group,
R³ denotes a hydrogen atom or a methyl group,
R⁴ denotes a carbamoyl group, a linear or branched (C₄ to C₁₂) alkylaminocarbonyl group, a linear or branched (C₄ to C₁₂) alkoxylcarbonyl group, a linear or branched (C₂ to C₁₂) acyloxy group, a (C₂ to C₄) hydroxyalkylcarbonyl group, or a phenyl group,
A¹ denotes a hydroxy group or an organic residue having at least one sulfonic acid group, which binds to the structural unit via an oxygen atom or an NH group, and
(b) at least one starch compound dispersed as solid particles, and
(c) at least one solvent, different from water, that is liquid at about 25° C. and about 1013 mbar, selected from at least one (C₂ to C₆) alkyl alcohol having at least one hydroxy group, and
(d) based on the total weight of the agent, about 0 to about 20 wt %, in particular about 0 to about 10 wt %, particularly preferably 0 to about 5 wt % water.

(B): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one anionic setting polymer comprising at least one structural unit of formula (I) and at least one structural unit of formula (II)

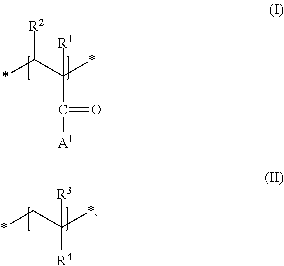

in which
R¹ and R² mutually independently denote a hydrogen atom or a methyl group, with the provision that R¹ and R² do not simultaneously denote a methyl group,
R³ denotes a hydrogen atom or a methyl group,
R⁴ denotes a carbamoyl group, a linear or branched (C₄ to C₁₂) alkylaminocarbonyl group, a linear or branched (C₄ to C₁₂) alkoxylcarbonyl group, a linear or branched (C₂ to C₁₂) acyloxy group, a (C₂ to C₄) hydroxyalkylcarbonyl group, or a phenyl group,
A¹ denotes a hydroxy group or an organic residue having at least one sulfonic acid group, which binds to the structural unit via an oxygen atom or an NH group, and
(b) about 1 to about 50 wt %, in particular from about 1.5 to about 35 wt % of at least one starch compound dispersed as solid particles, and
(c) about 49.9 to about 98.9 wt %, preferably about 60 to about 95 wt %, of at least one solvent, different from water, that is liquid at 25° C. and 1013 mbar, selected from at least one (C₂ to C₆) alkyl alcohol having at least one hydroxy group, and
(d) about 0 to about 20 wt %, in particular about 0 to about 10 wt %, particularly preferably 0 to about 5 wt % water.

(C): An agent for treating keratinic fibers, in particular human hair, containing
(a) at least one anionic setting polymer that comprises at least one structural unit of formula (I-2), at least one structural unit of formula (II), and at least one structural unit of formula (III)

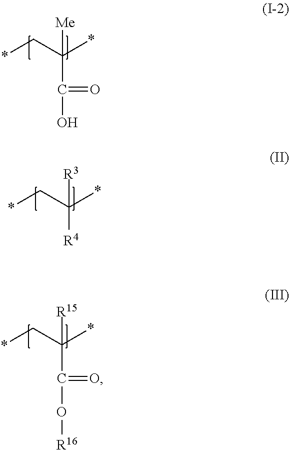

in which
R³ and R¹⁵ mutually independently denote a hydrogen atom or a methyl group,
R⁴ denotes a branched (C₄ to C₈) alkoxycarbonyl group, a linear (C₄ to C₈) alkoxycarbonyl group, a branched (C₄ to C₈) alkylaminocarbonyl group, a linear (C₄ to C₈) alkylaminocarbonyl group, or a phenyl group, and
R¹⁶ denotes a (C₁ to C₃) alkyl group (in particular a methyl group or an ethyl group), and
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and
(d) based on the total weight of the agent, about 0 to about 20 wt %, in particular about 0 to about 10 wt %, particularly preferably 0 to about 5 wt % water.

(D): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one anionic setting polymer that comprises at least one structural unit of formula (I-2), at least one structural unit of formula (II), and at least one structural unit of formula (III)

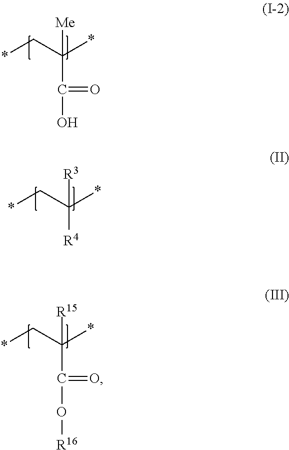

in which
R³ and R¹⁵ mutually independently denote a hydrogen atom or a methyl group,
R⁴ denotes a branched (C₄ to C₈) alkoxycarbonyl group, a linear (C₄ to C₈) alkoxycarbonyl group, a branched (C₄ to C₈) alkylaminocarbonyl group, a linear (C₄ to C₈) alkylaminocarbonyl group, or a phenyl group, and
R¹⁶ denotes a (C₁ to C₃) alkyl group (in particular a methyl group or an ethyl group), and
(b) about 1 to about 50 wt %, in particular about 1.5 to about 35 wt % of at least one starch compound dispersed as solid particles, and
(c) about 49.9 to about 98.9 wt %, preferably about 60 to about 95 wt %, of at least one solvent, different from water, that is liquid at 25° C. and 1013 mbar, selected from at least one (C₂ to C₆) alkyl alcohol having at least one hydroxy group, and
(d) about 0 to about 20 wt %, in particular about 0 to about 10 wt %, particularly preferably 0 to about 5 wt % water.

(E): An agent for treating keratinic fibers, in particular human hair, containing
(a) at least one anionic setting polymer that comprises at least one structural unit of formula (I-1), at least one structural unit of formula (II), and at least one structural unit of formula (III)

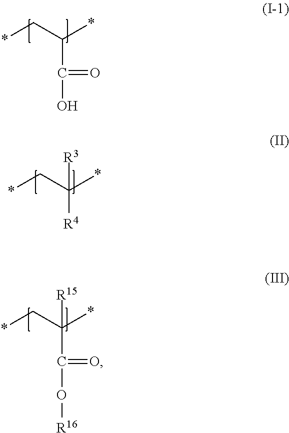

in which
R³ and R¹⁵ mutually independently denote a hydrogen atom or a methyl group,
R⁴ denotes a branched (C₄ to C₈) alkylaminocarbonyl group, a linear (C₄ to C₈) alkylaminocarbonyl group, or a phenyl group, and
R¹⁶ denotes a (C₁ to C₃) alkyl group (in particular a methyl group or an ethyl group), and
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and
(d) based on the total weight of the agent, about 0 to about 20 wt %, in particular about 0 to about 10 wt %, particularly preferably 0 to about 5 wt % water.

(F): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one anionic setting polymer that comprises at least one structural unit of formula (I-1), at least one structural unit of formula (II), and at least one structural unit of formula (III)

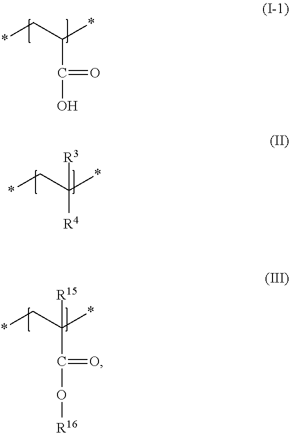

in which
R³ and R¹⁵ mutually independently denote a hydrogen atom or a methyl group,
R⁴ denotes a branched (C₄ to C₈) alkylaminocarbonyl group, a linear (C₄ to C₈) alkylaminocarbonyl group, or a phenyl group, and
R¹⁶ denotes a (C₁ to C₃) alkyl group (in particular a methyl group or an ethyl group),
(b) about 1 to about 50 wt %, in particular from about 1.5 to about 35 wt % of at least one starch compound dispersed as solid particles, and
(c) about 49.9 to about 98.9 wt %, preferably about 60 to about 95 wt %, of at least one solvent, different from water, that is liquid at 25° C. and 1013 mbar, selected from at least one (C₂ to C₆) alkyl alcohol having at least one hydroxy group, and
(d) about 0 to about 20 wt %, in particular about 0 to about 10 wt %, particularly preferably 0 to about 5% water.

(G): An agent for treating keratinic fibers, in particular human hair, containing
(a) at least one anionic setting polymer that contains at least one structural unit of formula (I-5) and at least one structural unit of formula (II-7)

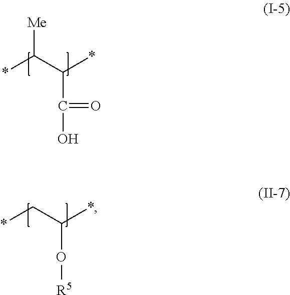

in which R⁵ denotes a (C₂ to C₁₂) acyl group (in particular acetyl or neodecanoyl), and
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and
(d) based on the total weight of the agent, about 0 to about 20 wt %, in particular about 0 to about 10 wt %, particularly preferably 0 to about 5 wt % water.

(H): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one anionic setting polymer that contains at least one structural unit of formula (I-5) and at least one structural unit of formula (II-7)

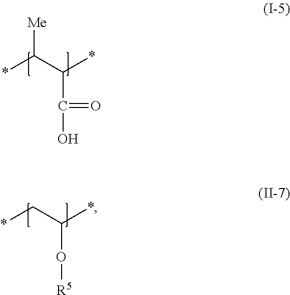

in which R⁵ denotes a (C₂ to C₁₂) acyl group (in particular acetyl or neodecanoyl), and
(b) about 1 to about 50 wt %, in particular from about 1.5 to about 35 wt % of at least one starch compound dispersed as solid particles, and
(c) about 49.9 to about 98.9 wt %, preferably about 60 to about 95 wt %, of at least one solvent, different from water, that is liquid at 25° C. and 1013 mbar, selected from at least one (C₂ to C₆) alkyl alcohol having at least one hydroxy group, and
(d) about 0 to about 20 wt %, in particular about 0 to about 10 wt %, particularly preferably 0 to about 5 wt % water.

(I): An agent for treating keratinic fibers, in particular human hair, containing
(a) at least amphoteric setting polymer selected from at least one copolymer of acrylic acid and at least one (C₁ to C₄) alkyl acrylate and at least one C₄ alkylaminoethyl methacrylate and at least one C₈ alkyl acrylamide, and
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and

(d) based on the total weight of the agent, about 0 to about 20 wt %, in particular about 0 to about 10 wt %, particularly preferably 0 to about 5 wt % water.

(J): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one amphoteric setting polymer selected from at least one copolymer of acrylic acid and at least one (C₁ to C₄) alkyl acrylate and at least one C₄ alkylaminoethyl methacrylate and at least one C₈ alkyl acrylamide, and
(b) about 1 to about 50 wt %, in particular from about 1.5 to about 35 wt % of at least one starch compound dispersed as solid particles, and
(c) about 10 to about 80 wt %, preferably about 15 to about 75 wt %, more preferably about 20 to about 70 wt %, and in particular about 35 to about 60 wt % of at least one solvent, different from water, that is liquid at 25° C. and 1013 mbar, selected from at least one (C₂ to C₆) alkyl alcohol having at least one hydroxy group, and
(d) about 0 to about 20 wt %, in particular about 0 to about 10 wt %, particularly preferably 0 to about 5 wt % water.

(K): An agent for treating keratinic fibers, in particular human hair, containing
(a) at least one anionic setting polymer that contains at least one structural unit of formula (I-5) and at least one structural unit of formula (II-7)

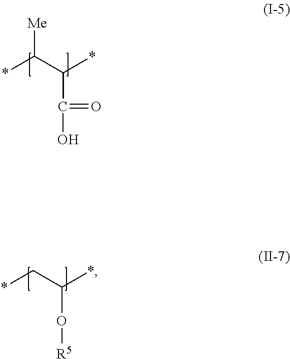

in which R⁵ denotes a (C₂ to C₁₂) acyl group (in particular acetyl or neodecanoyl) (in particular VA/Crotonates/Vinyl Neodecanoate Copolymer), and
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and
(d) based on the total weight of the agent, about 0 to about 1 wt % water.

(L): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one anionic setting polymer that contains at least one structural unit of formula (I-5) and at least one structural unit of formula (II-7)

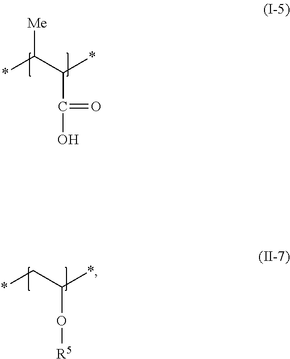

in which R⁵ denotes a (C₂ to C₁₂) acyl group (in particular acetyl or neodecanoyl) (in particular VA/Crotonates/Vinyl Neodecanoate Copolymer), and
(b) about 1 to about 50 wt %, in particular from about 1.5 to about 35 wt % of at least one starch compound dispersed as solid particles, and
(c) about 10 to about 80 wt %, preferably about 15 to about 75 wt %, more preferably about 20 to about 70 wt %, and in particular about 35 to about 60 wt % of at least one solvent, different from water, that is liquid at 25° C. and 1013 mbar, selected from at least one (C₂ to C₆) alkyl alcohol having at least one hydroxy group, and
(d) based on the total weight of the agent, about 0 to about 1 wt % water.

(M): An agent for treating keratinic fibers, in particular human hair, containing, based in each case on the total weight of the agent,
(a) at least one amphoteric setting polymer selected from at least one copolymer of acrylic acid and at least one (C₁ to C₄) alkyl acrylate and at least one C₄ alkylaminoethyl methacrylate and at least one C₈ alkyl acrylamide, and
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and
(d) based on the total weight of the agent, between 1 and 10 wt %, in particular about 1 to about 8 wt %, particularly preferably about 1 to about 5 wt % water,
(e) at least one (C₈ to C₂₀) monocarboxylic acid compound of formula (MC-1)

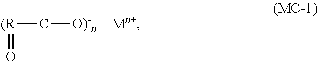

in which R denotes a (C₇ to C₁₉) alkyl group, Mⁿ⁺ denotes an n-valent cation, and n denotes the number 1, 2, or 3 (in particular 2).

(N): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one amphoteric setting polymer selected from at least one copolymer of acrylic acid and at least one (C₁ to C₄) alkyl acrylate and at least one C₄ alkylaminoethyl methacrylate and at least one C₈ alkyl acrylamide, and
(b) about 1 to about 50 wt %, in particular from about 1.5 to about 35 wt % of at least one starch compound dispersed as solid particles, and
(c) about 49.9 to about 98.9 wt %, preferably about 60 to about 95 wt %, of at least one solvent, different from water, that is liquid at 25° C. and 1013 mbar, selected from at least one (C₂ to C₆) alkyl alcohol having at least one hydroxy group, and
(d) based on the total weight of the agent, between about 1 and about 10 wt %, in particular about 1 to about 8 wt %, particularly preferably about 1 to about 5 wt % water,
(e) at least one (C₈ to C₂₀) monocarboxylic acid compound of formula (MC-1)

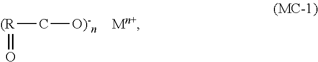

in which R denotes a (C₇ to C₁₉) alkyl group, Mⁿ⁺ denotes an n-valent cation, and n denotes the number 1, 2, or 3 (in particular 2).

(O): An agent for treating keratinic fibers, in particular human hair, containing
(a) at least one temporarily cationic setting polymer that comprises at least one structural unit of formula (M1) and at least one structural unit of formula (M2) and at least one structural unit of formula (M3)

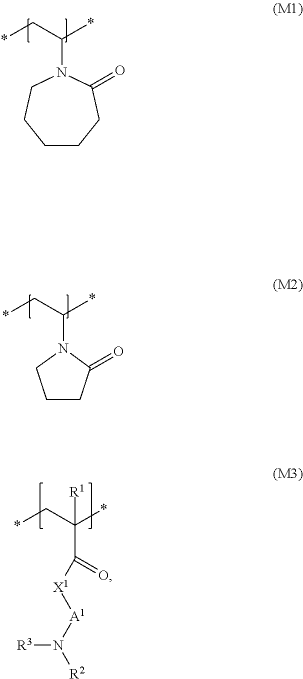

R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R⁴ mutually independently denote a (C₁ to C₄) alkyl group.
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and
(d) based on the total weight of the agent, about 0 to about 20 wt %, in particular about 0 to about 10 wt %, particularly preferably 0 to about 5 wt % water.

(P): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10.0 wt % of at least one temporarily cationic setting polymer that comprises at least one structural unit of formula (M1) and at least one structural unit of formula (M2) and at least one structural unit of formula (M3)

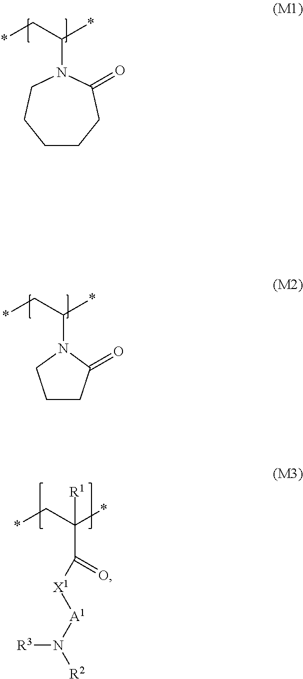

R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R⁴ mutually independently denote a (C₁ to C₄) alkyl group, and
(b) about 1 to about 50 wt %, in particular from about 1.5 to about 35 wt % of at least one starch compound dispersed as solid particles, and
(c) about 49.9 to about 98.9 wt %, preferably about 60 to about 95 wt %, of at least one solvent, different from water, that is liquid at 25° C. and 1013 mbar, selected from at least one (C₂ to C₆) alkyl alcohol having at least one hydroxy group, and
(d) about 0 to about 20 wt %, in particular about 0 to about 10 wt %, particularly preferably about 0 to about 5 wt % water.

(Q): An agent for treating keratinic fibers, in particular human hair, containing
(a) as a setting polymer at least a combination of
   (i) at least one temporarily cationic setting polymer comprising at least one structural unit of formula (M1) and at least one structural unit of formula (M2) and at least one structural unit of formula (M3)

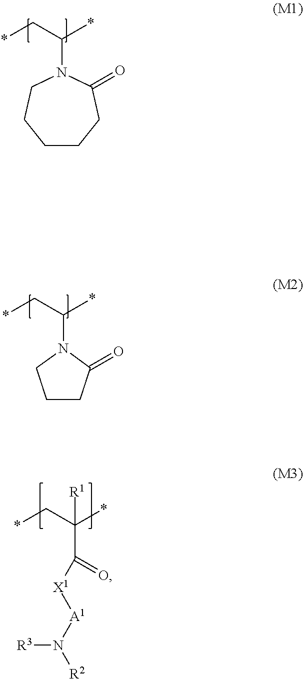

R₁ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R⁴ mutually independently denote a (C₁ to C₄) alkyl group,
and
(ii) at least one amphoteric setting polymer comprising at least one structural unit of formula (I) and at least one structural unit of formula (II)

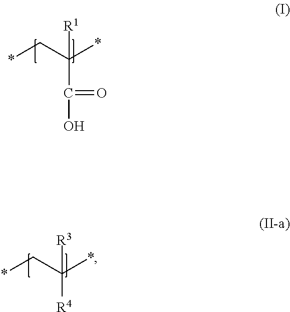

in which
R¹ and R³ mutually independently denote a hydrogen atom or a methyl group,
R⁴ denotes a linear or branched (C₄ to C₁₂) alkylaminoethylaminocarbonyl group, a linear or branched (C₄ to C₁₂) alkylaminopropylaminocarbonyl group, a linear or branched (C₄ to C₁₂) alkylaminoethyloxycarbonyl group, a linear or branched (C₄ to C₁₂) alkylaminopropyloxycarbonyl group, and
(b) at least one starch compound dispersed as solid particles, and
(c) at least one solvent, different from water, that is liquid at 25° C. and 1013 mbar, selected from at least one (C₂ to C₆) alkyl alcohol having at least one hydroxy group, and
(d) based on the total weight of the agent, about 0 to about 20 wt %, in particular about 0 to about 10 wt %, particularly preferably 0 to about 5 wt % water.

(R): An agent for treating keratinic fibers, in particular human hair, containing, based in each case on the total weight of the agent,
(a) about 0.1 wt % to about 20.0 wt %, particularly preferably from about 0.2 wt % to about 15.0 wt %, very particularly preferably from about 0.5 wt % to about 10 wt % of a combination of
   (i) at least one temporarily cationic setting polymer comprising at least one structural unit of formula (M1) and at least one structural unit of formula (M2) and at least one structural unit of formula (M3)

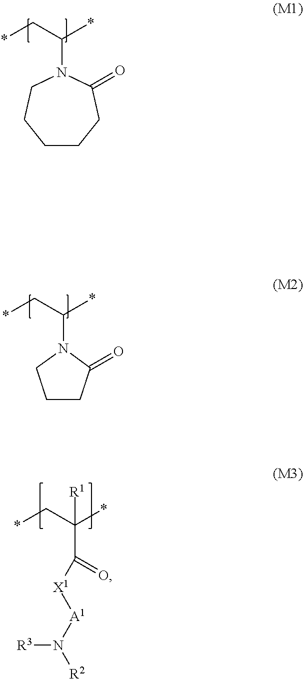

R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R⁴ mutually independently denote a (C₁ to C₄) alkyl group,
and
(ii) at least one amphoteric setting polymer comprising at least one structural unit of formula (I) and at least one structural unit of formula (II)

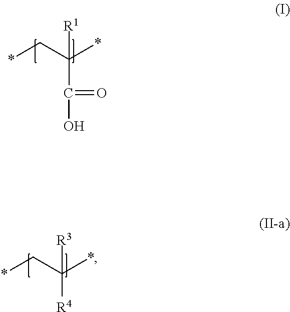

in which
R¹ and R³ mutually independently denote a hydrogen atom or a methyl group,
R⁴ denotes a linear or branched (C₄ to C₁₂) alkylaminoethylaminocarbonyl group, a linear or branched (C₄ to C₁₂) alkylaminopropylaminocarbonyl group, a linear or branched (C₄ to C₁₂) alkylaminoethyloxycarbonyl group, a linear or branched (C₄ to C₁₂) alkylaminopropyloxycarbonyl group, and
(b) about 1 to about 50 wt %, in particular from about 1.5 to about 35 wt % of at least one starch compound dispersed as solid particles, and
(c) about 49.9 to about 98.9 wt %, preferably about 60 to about 95 wt %, of at least one solvent, different from water, that is liquid at 25° C. and 1013 mbar, selected from at least one (C₂ to C₆) alkyl alcohol having at least one hydroxy group, and
(d) about 0 to about 20 wt %, in particular about 0 to about 10 wt %, particularly preferably 0 to about 5 wt % water.

For the embodiments (A) to (N) and (Q) to (R) it is preferred if the cosmetic agent additionally contains at least one alkanolamine.

The preferred embodiments of the features recited above also apply mutatis mutandis to the embodiments (A) to (R).

The agents preferably have a pH from 2 to 11. The pH range is particularly preferably between 2 and 8. The indications as to pH refer, for purposes of this document, to the pH at 25° C. unless otherwise noted.

Alkyl esters of hydroxy acids can be added to the agents according to the present invention in order to improve the feel of the hair. Agents preferred according to the present invention are characterized in that they contain, based in each case on the weight of the total ready-to-use agent, about 0.05 to about 5 wt %, preferably about 0.1 to about 2.5 wt %, and in particular about 0.15 to about 0.5 wt % of at least one alkyl ester of a hydroxy acid.

Very particularly preferred alkyl esters of hydroxy acids are the (C₂ to C₆) trialkyl citrates. It is therefore preferred according to the present invention if the agents additionally contain at least one compound of formula (E)

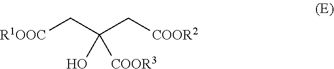

in which
R′, R², and R³ mutually independently denote a (C₂ to C₆) alkyl group.

Examples of a (C₂ to C₆) alkyl group according to formula (E) are methyl, ethyl, isopropyl, n-propyl, n-butyl, sec-butyl, isobutyl, tert-butyl, n-pentyl, neopentyl, isopentyl, n-hexyl.

Triethyl citrate emerges as a particularly preferred compound of formula (E).

The agent according to the present invention contains the compounds of formula (E) preferably in a quantity from about 0.01 to about 1 wt %, in particular from about 0.05 to about 0.3 wt %, based in each case on the weight of the total agent. Particularly preferred agents according to the present invention are characterized in that they contain, based in each case on the weight of the total ready-to-use agent, about 0.05 to about 5 wt %, preferably about 0.1 to about 2.5 wt %, and in particular about 0.15 to about 0.5 wt % triethyl citrate.

The agents according to the present invention can furthermore contain the adjuvants and additives that are usually added to conventional styling agents.

Additional care-providing substances are to be recited in particular as suitable adjuvants and additives.

The agent can contain as a care-providing substance, for example, at least one protein hydrolysate and/or one of its derivatives.

Protein hydrolysates are product mixtures obtained by the acid-, base-, or enzyme-catalyzed breakdown of proteins. The term “protein hydrolysates” is also understood according to the present invention to mean total hydrolysates as well as individual amino acids and derivatives thereof, as well as mixtures of different amino acids. Polymers constructed from amino acids and amino acid derivatives are also understood according to the present invention under the term “protein hydrolysates.”

Beta-amino acids and derivatives thereof can of course also be used according to the present invention. The molecular weight of the protein hydrolysates usable according to the present invention is between about 75 (the molecular weight of glycine) and 200,000; the molecular weight is preferably equal to about 75 to 50,000 dalton, and very particularly preferably to about 75 to 20,000 dalton.

According to the present invention, protein hydrolysates of both vegetable and animal origin, or of marine or synthetic origin, can be used.

Although the use of protein hydrolysates as such is preferred, amino acid mixtures obtained in other ways can also optionally be used instead of them.

The protein hydrolysates can be contained in the agents according to the present invention, for example, in concentrations from about 0.01 wt % to about 20 wt %, preferably from about 0.05 wt % to about 15 wt %, and very particularly preferably in quantities from about 0.05 wt % to about 5 wt %, based in each case on the total utilization preparation.

The agent according to the present invention can further contain at least one vitamin, provitamin, vitamin precursor, and/or one of their derivatives as a care-providing substance.

Those vitamins, provitamins, and vitamin precursors which are usually assigned to groups A, B, C, E, F, and H are preferred according to the present invention.

The agents according to the present invention preferably contain vitamins, provitamins, and vitamin precursors from groups A, B, C, E and H. Panthenol, pantolactone, pyridoxine and derivatives thereof, as well as nicotinic acid amide and biotin, are particularly preferred.

Like the addition of glycerol and/or propylene glycol, the addition of panthenol increases the flexibility of the polymer film formed upon utilization of the agent according to the present invention. If a particularly flexible hold is desired, the agents according to the present invention can thus contain panthenol instead of or in addition to glycerol and/or propylene glycol. In a preferred embodiment the agents according to the present invention contain panthenol, preferably in a quantity from about 0.05 to about 10 wt %, particularly preferably about 0.1 to about 5 wt %, based in each case on the total agent.

The agents according to the present invention can furthermore contain at least one plant extract as a care-providing substance. These extracts are usually produced by extraction of the entire plant. In individual cases, however, it may also be preferred to produce the extracts exclusively from blossoms and/or from leaves of the plant. According to the present invention the extracts from green tea, oak bark, nettle, witch hazel, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root are especially preferred.

The plant extracts can be used according to the present invention in both pure and diluted form. If they are used in diluted form, they usually contain approx. about 2 to about 80 wt % active substance and, as a solvent, the extraction agent or extraction agent mixture used to recover them. It can further be preferred to use in the agents according to the present invention mixtures of several, in particular two, different plant extracts. The agents according to the present invention contain these care-providing substances preferably in quantities from about 0.001 to about 2, in particular from about 0.01 to about 0.5 wt %, based in each case on the total utilization preparation.

It has been found that the addition of a UV filter allows both the agents themselves, and the treated fibers, to be protected from damaging influences of UV radiation. Surprisingly, the shine of the fibers treated with the agents according to the present invention also becomes improved and more long-lasting. At least one UV filter is therefore preferably added to the agent. The UV filters are contained usually in quantities from about 0.01 to about 5 wt %, based on the total utilization preparation. Quantities from about 0.1 to about 2.5 wt % are preferred. Agents preferred according to the present invention contained, based in each case on the weight of the total ready-to-use agent, about 0.05 to about 5 wt %, preferably about 0.1 to about 2.5 wt %, and in particular about 0.15 to about 0.5 wt % UV filter substances.

The suitable UV filters are not subject to any general restrictions in terms of their structure and their physical properties. Instead, all UV filters usable in the cosmetics sector, whose absorption maximum lies in the UVA (315 to 400 nm) UVB (280 to 315 nm), or UVC (<280 nm) regions, are suitable. UV filters having an absorption maximum in the UVB region, in particular in the region from approximately 280 to approximately 300 nm, are particularly preferred.

The UV filters preferred according to the present invention can be selected, for example, from substituted benzophenones, p-aminobenzoic acid esters, diphenylacrylic acid esters, cinnamic acid esters, salicylic acid esters, benzimidazoles, cinnamates, and o-aminobenzoic acid esters.

Those UV filters whose molar extinction coefficient at the absorption maximum is above 15,000, in particular above 20,000, are preferred.

Very particularly preferred agents according to the present invention contain, based in each case on the weight of the total ready-to-use agent, about 0.05 to about 5 wt %, preferably about 0.1 to about 2.5 wt %, and in particular about 0.15 to about 0.5 wt % benzophenone-4 and/or isoamyl p-methoxycinnamate.

Besides the components recited, the agents can furthermore contain all active agents, additives, and adjuvants known for such preparations.

Further active agents, additives, and adjuvants are, for example
structuring agents such as maleic acid and lactic acid,
perfume oils, dimethylisosorbide, and cyclodextrins,
defoamers such as silicones,
dyes for coloring the agent,
anti-dandruff active agents such as piroctone olamine, zinc omadine, and climbazol,
consistency agents such as sugar esters, polyol esters, or polyolalkyl ethers,
complexing agents such as EDTA, NTA, β-alaninediacetic acid, and phosphonic acids,
swelling and penetration substances such as carbonates, hydrogen carbonates, guanidines, ureas, and primary, secondary, and tertiary phosphates,
opacifiers such as latex, styrene/PVP and styrene/acrylamide copolymers,
luster agents such as ethylene glycol mono- and distearate as well as PEG-3 distearate,
preservatives,
emulsifier agents,
antioxidants.

With regard to further optional components as well as the quantities of those components used, reference is made expressly to the relevant manuals known to one skilled in the art.

The agents according to the present invention can be formulated in all forms usual for styling agents, for example in the form of solutions that can be applied onto the hair as a lotion or as a pump spray or aerosol spray, or of other preparations that are suitable for use on the hair.

The agents according to the present invention are configured preferably as a pump spray, aerosol spray, pump foam, or aerosol foam. For this, the agents according to the present invention are packaged in a delivery apparatus that is either a pressurized-gas container additionally filled with a propellant (“aerosol container”) or a non-aerosol container. The pressurized-gas containers with which a product is distributed through a valve as a result of the internal gas pressure of the container are referred to by definition as “aerosol containers.” A “non-aerosol container” is defined, conversely to the “aerosol” definition, as a vessel under standard pressure with which a product is distributed by means of mechanical action by way of a pump system or squeeze system.

The agents according to the present invention are present particularly preferably as an aerosol hair foam or aerosol hair spray.

Agents according to the present invention that are present in the form of an aerosol product can be manufactured in usual fashion. As a rule all the constituents of the agent according to the present invention are introduced into a suitable pressure-tight container. The latter is then sealed with a valve. Lastly, the desired quantity of propellant is introduced using conventional techniques. A second subject of the present invention is therefore an apparatus for reshaping keratin-containing fibers, in particular human hair, comprising an aerosol delivery apparatus, in particular an aerosol spray apparatus, and a composition contained therein that comprises, based on its weight:
about 1 to about 89 wt %, preferably about 5 to about 50 wt %, and in particular about 10 to about 40 wt % of a cosmetic agent of the first subject of the present invention, and
about 20 to about 99 wt %, preferably about 50 to about 95 wt %, and in particular about 60 to about 90 wt % of at least one propellant.

The agents (A) to (N) preferred according to the present invention are in turn preferably suitable. All preferred features (vide supra) of the constituents contained therein are in turn preferred, mutatis mutandis.

In the embodiment as an aerosol spray, propellants suitable according to the present invention are selected, for example, from N₂O, dimethyl ether, CO₂, air, alkanes having 3 to 5 carbon atoms such as propane, n-butane, isobutane, n-pentane, and isopentane, and mixtures thereof. Dimethyl ether, propane, n-butane, isobutane, and mixtures thereof are preferred. In accordance with a preferred embodiment, the aforesaid alkanes, mixtures of the aforesaid alkanes, or mixtures of the aforesaid alkanes with dimethyl ether are used as the only propellant. The invention also expressly encompasses, however, the concurrent use of propellants of the fluorochlorocarbon type, but in particular fluorocarbons.

The propellant is contained in the agents according to the present invention, in the embodiment as an aerosol spray, preferably in a quantity from about 30 to about 60 wt %, based on the weight of the total agent.

Very particularly preferably, dimethyl ether or mixtures of propane and butane are used as the only propellant, at a weight ratio of propane to butane from about 20 to about 80 to about 15 to about 85. The mixtures are in turn used in the agents according to the present invention preferably in a quantity from about 30 to about 55 wt %, based on the weight of the total agent. “Butane” is understood according to the present invention as n-butane, isobutane, and mixtures of n-butane and isobutane.

Most preferably, dimethyl ether is used as the only propellant.

For a given spray apparatus, the sizes of the aerosol droplets and the respective size distribution can be adjusted by way of the quantitative ratio of propellant to the other constituents of the preparations.

The spray rate of the sprays according to the present invention is preferably about 6.5 to about 10.0 g/10 s.

Particularly preferred agents according to the present invention (aerosol sprays) are packaged in an aerosol container having a stem valve with a stem orifice about 0.27 to about 0.35 mm in diameter. Such valves are marketed, for example, by the Coster company as valves of the KE or KEN type.

In the embodiment as an aerosol foam, propellants suitable according to the present invention are selected, for example, from N₂O, dimethyl ether, CO₂, air, alkanes having 3 to 5 carbon atoms such as propane, n-butane, isobutane, n-pentane, and isopentane, and mixtures thereof. The invention also expressly encompasses, however, the concurrent use of propellants of the fluorochlorocarbon type, but in particular fluorocarbons.

In accordance with the embodiment of an aerosol foam, the aforesaid alkanes, mixtures of the aforesaid alkanes, or mixtures of the aforesaid alkanes with dimethyl ether are preferably used as the only propellant. Particularly preferred propellants are dimethyl ether, propane, n-butane, isobutane, and mixtures thereof.

For a given spray apparatus, the sizes of the foam bubbles and the respective size distribution can be adjusted by way of the quantitative ratio of propellant to the other constituents of the preparations.

When conventional aerosol containers are used, aerosol foam products contain the propellant preferably in quantities from about 1 to about 35 wt %, based on the total product. Quantities from about 2 to about 30 wt %, in particular from about 3 to about 15 wt %, are particularly preferred.

Isopentane is preferably suitable as a propellant for foaming gel-type agents in a two-chamber aerosol container, said propellant being incorporated into the agents according to the present invention and being packaged in the first chamber of the two-chamber aerosol container. Packaged in the second chamber of the two-chamber aerosol container is at least one further propellant, different from isopentane, that builds up in the two-chamber aerosol container a higher pressure than the isopentane. The propellants of the second chamber are preferably selected from N₂O, dimethyl ether, CO₂, air, alkanes having 3 or 4 carbon atoms (such as propane, n-butane, isobutane), as well as mixtures thereof.

A further subject of the invention is a method for treating keratin-containing fibers, in particular human hair, in which (preferably using an aerosol delivery apparatus) an agent in accordance with the first subject of the invention is applied onto the keratin-containing fibers and the keratin-containing fibers are brought into shape, and that shape is retained using the agent of the first subject of the invention, with the provision that after application of the agent according to the present invention, the agent according to the present invention remains on the keratin-containing fibers, i.e. is not rinsed out again. The delivery apparatuses resp. aerosol products recited above are considered preferred according to the present invention.

The agents according to the present invention and products that contain said agents, in particular aerosol hair sprays and aerosol hair foams, are notable in particular for the fact that they impart a strong hold and volume to treated hair.

A further subject of the invention is a method for remodeling an imposed shape of a fiber collective of keratin-containing fibers (in particular for remodeling styled head hair), in which
(i) the fibers (in particular the hair) are dry before remodeling begins, and already have a shape retained by utilization of a cosmetic agent of the first subject of the invention,
(ii) these fibers (in particular the hair) are brought into a new shape, and
(iii) this new shape is retained again by means of the cosmetic agent already applied before step (i) onto the fibers (in particular onto the hair) in order to retain the shape,
   with the provision that no application of a further cosmetic agent occurs in order to retain the new shape for purposes of (iii).

A further subject of the invention is the use of a cosmetic agent of the first subject of the invention to retain a shape of a fiber collective of keratin-containing fibers, in particular to retain a hairstyle.

A further subject of the invention is the use of a cosmetic agent of the first subject of the invention to remodel a remodelable shape, retained with said cosmetic agent, of a fiber collective of keratin-containing fibers, in particular to remodel a remodelable hairstyle retained with said cosmetic agent.

### EXAMPLES

The following formulations were prepared by mixing the raw materials indicated, and were decanted into an aerosol can having a model KE valve of the Coster company and a model V06 “Standard micromist insert” swirl nozzle of the Coster company. The aerosol cans were sealed with the valve, and lastly the corresponding propellant (here dimethyl ether) was added. The following quantitative indications are understood, unless otherwise noted, as percentages by weight.

| Raw material | E1 (wt %) | E2 (wt %) |
|---|---|---|
| Ethanol | to 100 | to 100 |
| Water | 1.20 | 0.03 |
| Amphomer ¹ | 0.50 | — |
| Resyn 28-2930 ² | — | 0.5 |
| 2-Amino-2-methylpropan-1-ol | 0.09 | 0.05 |
| Rice Starch D.S.A. 7 ³ | 0.40 | 0.4 |
| Magnesium distearate | 0.5 | — |
| Dimethyl ether | 70.0 | 70.0 |

¹ INCI name: Octylacrylamide/Acrylates/Butylaminoethyl Methacrylate Copolymer (100% active substance, powdered) (Akzo Nobel)
² INCI name: VA/Crotonates/Vinyl Neodecanoate Copolymer (copolymer of vinyl acetate, vinyl neodecanoate, and crotonic acid, 98-100% active substance) (Akzo Nobel)
³ INCI name: Oryza Sativa (Rice) Starch, Cetrimonium Chloride (Agrana)

1. A cosmetic agent for treating keratin-containing fibers, in particular human hair, containing
(a) at least one setting polymer,
(b) at least one starch compound dispersed as solid particles,
(c) at least one solvent, different from water, that is liquid at about 25° C. and about 1013 mbar, and
(d) based on the total weight of the agent, about 0 to about 20 wt % water.

2. The agent according to claim 1, characterized in that said setting polymer is selected from one or more representatives of the group that is constituted from anionic setting polymers, amphoteric setting polymers, temporarily cationic setting polymers, and nonionic setting polymers.

3. The agent according to claim 2, characterized in that at least one anionic setting polymer comprising at least one structural unit of formula (I) and at least one structural unit of formula (II) 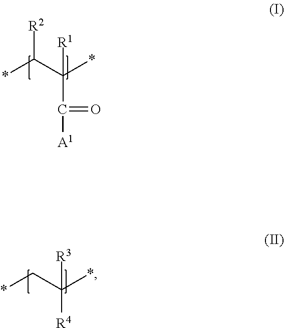
in which
R¹ and R² mutually independently denote a hydrogen atom or a methyl group, with the provision that R¹ and R² do not simultaneously denote a methyl group,
R³ denotes a hydrogen atom or a methyl group,
R⁴ denotes a carbamoyl group, a linear or branched (C₄ to C₁₂) alkylaminocarbonyl group, a linear or branched (C₄ to C₁₂) alkoxylcarbonyl group, a linear or branched (C₂ to C₁₂) acyloxy group,
A¹ denotes a hydroxy group or an organic residue having at least one sulfonic acid group, which binds to the structural unit via an oxygen atom or an NH group, is contained as said setting polymer.

4. The agent according to claim 3, characterized in that at least one amphoteric setting polymer comprising at least one structural unit of formula (I) and at least one structural unit of formula (II-a) 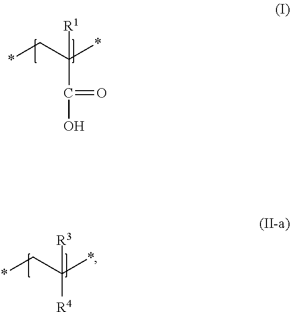
in which
R¹ and R³ mutually independently denote a hydrogen atom or a methyl group,
R⁴ denotes a linear or branched (C₄ to C₁₂) alkylaminoethylaminocarbonyl group, a linear or branched (C₄ to C₁₂) alkylaminopropylaminocarbonyl group, a linear or branched (C₄ to C₁₂) alkylaminoethyloxycarbonyl group, a linear or branched (C₄ to C₁₂) alkylaminopropyloxycarbonyl group, is contained as said setting polymer.

5. The agent according to claim 4, characterized in that the amphoteric setting polymer is selected from at least one copolymer of acrylic acid, at least one (C₁ to C₄) alkyl acrylate, at least one C₄ alkylaminoethyl methacrylate, and at least one C₈ alkyl acrylamide.

6. The agent according to one of claim 5, characterized in that it contains as said setting polymer at least one temporarily cationic setting polymer that comprises at least one structural unit of formula (M1) and at least one structural unit of formula (M2) and at least one structural unit of formula (M3) 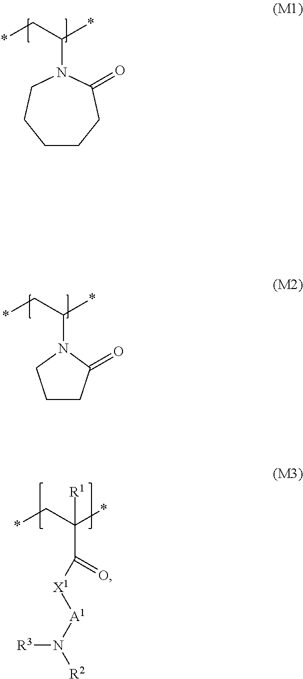
R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R³ mutually independently denote a (C₁ to C₄) alkyl group.

7. The agent according to claim 6, characterized in that it contains said setting polymers, based on the total weight of the agent, in a quantity from about 0.1 wt % to about 20.0 wt %.

8. The agent according to claim 7, characterized in that said starch compound dispersed as solid particles is contained in a quantity from about 1 to about 50 wt % based on the total weight of the agent.

9. The agent according to claim 8, characterized in that said solvent comprises at least one (C₂ to C₆) alkyl alcohol having at least one hydroxy group.

10. The agent according to claim 9, characterized in that based on the total weight of the agent, about 49.9 to about 98.9 wt % of said solvent is contained.

11. The agent according to claim 10, characterized in that it additionally contains at least one alkanolamine.

12. The agent according to claim 11, characterized in that it contains, based on its total weight, water in a quantity between about 1.0 and about 20.0 wt %, and additionally at least one (C₈ to C₂₀) monocarboxylic acid compound of formula (MC-1) 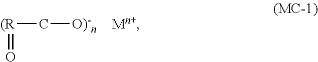
in which R denotes a (C₇ to C₁₉) alkyl group, Mⁿ⁺ denotes an n-valent cation, and n denotes the number 1, 2, or 3.

13. The agent according to claim 12, characterized in that at least one (C₈ to C₂₀) monocarboxylic acid compound of formula (MC-1) is selected from among ammonium stearate, calcium distearate, magnesium distearate, zinc distearate, ammonium isostearate, calcium diisostearate, magnesium diisostearate, zinc diisostearate, ammonium palmitate, calcium dipalmitate, magnesium dipalmitate, zinc dipalmitate, or mixtures thereof.

14. An apparatus for reshaping keratinic fibers comprising an aerosol delivery apparatus and a composition contained therein that comprises, based on its weight,
about 1 to about 89 wt % of a cosmetic agent containing at least one setting polymer, at least one starch compound dispersed as solid particles, at least one solvent, different from water, that is liquid at about 25° C. and about 1013 mbar, and about 0 to about 20 wt % water based on the total weight of the agent, and
about 20 to about 99 wt % of at least one propellant.

15. A method for remodeling an imposed shape of a fiber collective of keratin-containing fibers, in which
(i) the fibers are dry before remodeling begins, and already have a shape retained by utilization of a cosmetic agent containing at least one setting polymer, at least one starch compound dispersed as solid particles, at least one solvent, different from water, that is liquid at about 25° C. and about 1013 mbar, and about 0 to about 20 wt % water based on the total weight of the agent,
(ii) the fibers are brought into a new shape, and
(iii) the new shape is retained by means of the cosmetic agent already applied before step (i) onto the fibers in order to retain the shape,
with the provision that no application of a further cosmetic agent occurs in order to retain the new shape for purposes of (iii).
